(19) United States
(12) Patent Application Publication
(10) Pub. No.: US 2024/0217923 A1
(43) Pub. Date: Jul. 4, 2024

(54) A DIISOCYANATE STABILIZER, USE THEREOF, AND DIISOCYANATE COMPOSITION

(71) Applicants: BASF SE, Ludwigshafen Am Rhein (DE); BASF (China) Company Limited, Shanghai (CN)

(72) Inventors: Guo Liang Yuan, Shanghai (CN); Michael Ishaque, Shanghai (CN); Shun Ying Jin, Shanghai (CN); Ke Wang, Shanghai (CN); Yi Zhou Zheng, Shanghai (CN)

(21) Appl. No.: 18/555,812

(22) PCT Filed: Mar. 28, 2022
(86) PCT No.: PCT/EP2022/058138

§ 371 Date: Oct. 17, 2023

(30) Foreign Application Priority Data
Apr. 26, 2021 (WO) PCT/CN2021/089934

(51) Int. Cl. C07C 263/18

(52) U.S. Cl. CPC C07C 263/18

(57) ABSTRACT

The present disclosure relates to a diisocyanate stabilizer, use of the diisocyanate stabilizer for stabilizing diisocyanate, and a diisocyanate composition comprising the stabilizer. The diisocyanate stabilizer comprises a sterically hindered phenol other than butylated hydroxytoluene, a thioether, and a phosphite other than triphenyl phosphite. The present disclosure aims to provide a diisocyanate stabilizer which can make diisocyanates maintain stable during long-term storage and heating condition.

### TECHNICAL FIELD

The present disclosure relates to a diisocyanate stabilizer, use of the diisocyanate stabilizer for stabilizing diisocyanate, and a diisocyanate composition comprising said stabilizer.

### BACKGROUND

Diisocyanates are important intermediates in organic synthesis, for example, in the preparation of polyurethane resins. However, due to the high reactivity of isocyanate groups, diisocyanates tend to turn yellow and/or become turbid due to self-polymerization during storage which thereby affects its application in downstream industries. In order to prevent diisocyanates from turning yellow and/or becoming turbid, skilled person in the art usually adds a certain amount of antioxidants to the diisocyanates.

For example, U.S. Pat. No. 3,555,072 discloses a process for inhibiting discolouration and turbidity formation in toluene diisocyanate (TDI) which comprises adding thereto a stabilizing amount of thioether compounds.

However, the long-term thermal stability of the TDI solution obtained according to U.S. Pat. No. 3,555,072 is poor. In order to prolong the thermal stability, skilled person in the art may increase the amount of the thioether but this in turn will cause an odor issue to TDI which is also unacceptable.

Therefore, there is still a need in the art to provide a stabilizer for stabilizing diisocyanates, which enables diisocyanates to maintain colorless and transparent after a long storage period.

### SUMMARY OF THE INVENTION

The present disclosure provides a diisocyanate stabilizer which solves or at least partly solves above problems or other potential problems for stabilizing diisocyanates that exist in prior art.

In specific, in a first aspect, the present disclosure provides a diisocyanate stabilizer, comprising a sterically hindered phenol other than butylated hydroxytoluene, a thioether and a phosphite other than triphenyl phosphite.

In some embodiments, the sterically hindered phenol is present in an amount of 10 to 90 wt %, the thioether is present in an amount of 5 to 80 wt %, and the phosphite is present in an amount of 5 to 80 wt %, in each case based on the total weight of the stabilizer.

In other embodiments, the diisocyanate stabilizer consists of a sterically hindered phenol, a thioether and a phosphite.

Inventors of the present disclosure surprisingly found that sterically hindered phenol, thioether and phosphite can work synergistically to effectively prevent diisocyanates from self-polymerization. By combining sterically hindered phenol, thioether and phosphite together, the present disclosure provides a stabilizer, which can prevent diisocyanates from turning yellow and/or becoming turbid during long-term storage and heating condition. The stabilizer of the present disclosure has effectively solved the problems that the diisocyanates are easy to turn yellow and/or become turbid during long-term storage and heating condition.

In some preferable embodiments, the diisocyanate stabilizer has a melting point of lower than 50° C., preferably lower than 20° C. For example, the diisocyanate stabilizer has a melting point of lower than 50° C., 45° ° C., 40° C., 35° C., 30° C., 25° C., 20° C. or 10° C. In these preferable embodiments, the diisocyanate stabilizer is liquid at operational temperature (typically at 45° C. to 50° C.) to facilitate automatic production, which can thereby improve production efficiency. Further, when the diisocyanate stabilizer has a melting point of lower than 20° C. and is therefore liquid even below 20° C., it additionally facilitates the transportation of the diisocynate stabilizer at low temperature.

In a second aspect, the present disclosure provides use of the stabilizer according to the first aspect of the present disclosure for stabilizing diisocyanate.

In a third aspect, the present disclosure provides a composition comprising:
(A) a diisocyanate, and
(B) the diisocyanate stabilizer according to the first aspect of the present disclosure.

Inventors of the present disclosure surprisingly found that the obtained diisocyanate composition can maintain (substantively) transparent and colourless during long-term storage and heating condition which significantly broadens the application areas of diisocyanates. Accordingly, the diisocyanate composition of the present disclosure can be qualified for applications having high requirements in colour and transparency aspects.

Compared to prior art, the advantageous technical effects brought by the diisocyanate stabilizer of the present disclosure to diisocyanate during long-term storage and heating condition (e.g. melting process having a temperature of around 100° C.) include but not limited to:
(1) The diisocyanate composition has a lighter colour;
(2) The diisocyanate composition has a higher transparency; and
(3) The diisocyanate composition maintains a lighter colour and higher transparency.

It should be understood that the part of summary of the invention is neither intended to determine key or basic features of the present disclosure nor to limit the scope of the present disclosure. Other features of the present disclosure will become easily understandable in accordance with the below description.

### DETAILED DESCRIPTION OF THE INVENTION

In the below description, further explanations to the present disclosure are made with reference to embodiments so as to facilitate sufficient understanding for skilled person in the art. It should be understood that these embodiments are provided merely for better understanding the subject matter of the present disclosure, not for making any limitations to the protection scope, applicability or embodiments as described in the present claims set. It should be understood that, skilled person in the art can omit, replace, or add various technical features to each embodiment based on actual needs, subject to the premise of without departing from the spirit of the present disclosure. In addition, technical features described in some embodiments can be combined with technical features described in other embodiments.

Unless defined otherwise, all technical and scientific terms used herein have the same meanings as commonly understood by skilled person in the art to which the present disclosure belongs.

In the present disclosure, terms “comprise”, “comprising” and various variants thereof can be understood as open-ended terms, which means “include but are not limited to”; in contrast, the term “consisting of” and various variants thereof excludes any component, step or procedure not specifically listed; the term “one embodiment” can be understood as “at least one embodiment”; the term “another embodiment” may be understood as “at least one other embodiment.” Other terms that may appear but are not mentioned here, unless explicitly stated, should not be interpreted or limited in a manner that is contrary to the concept on which the embodiments of the present disclosure are based.

Throughout the present disclosure, expressions “a”, “an”, “the” and “one or more” are used interchangeably and are intended to include both the plural and the singular except in cases where the singular alone is explicitly specified or is clearly indicated by the context. When the singular alone is intended for, the term “one” is typically used. The term “or” is generally intended to include the sense of “and/or” unless the content clearly dictates otherwise. “Preferred”, “preferable” and “preferably” as used herein refer to embodiments of the present disclosure that may bring certain advantages under certain situations. However, other embodiments may also be preferred, under the same situations. Further, the recitation of one or more preferred embodiments does not mean that other embodiments are not useful and is not intended to exclude other embodiments from the scope of the present disclosure.

All percentages, parts and ratios are by weight, unless otherwise specified. And the recitation of numerical ranges by end values includes all numbers subsumed within that range (e.g., 5 to 10 includes 5, 5.1, 5.2, 5.55, 6, 6.5, 7, 7.5, 8, 8.5, 9, 9.5, 10).

In the present disclosure, “diisocyanate stabilizer” refers to a mixture/composition that is able to stabilize diisocyanate against internal or external influence factors. In terms of internal factors, as discussed above, diisocyanate tends to self-polymerize due to high reactive groups. In terms of external factors, diisocyanate is easily affected by heat, light and etc. The diisocyanate stabilizer is provided to stabilize diisocyanate in terms of colour stability, transparency and etc. Said diisocyanate stabilizer enables the diisocyanate to maintain colourless and transparent during long-term storage and heating condition.

Throughout the present disclosure, expressions “phosphite”, “phosphites” are used interchangeably and are intended to include both the plural and the singular except in cases where the singular alone is explicitly specified or is clearly indicated by the context.

The term “glycol” is an aliphatic diol containing two hydroxyl groups (—OH groups) attached to different carbon atoms.

The term “colour stability” means that the colour of the diisocyanate composition has no change or only slight change during long-term storage, even under heating conditions. For example, the diisocyanate composition of the present disclosure is able to maintain colourless or light yellow during long-term storage and heating condition.

The term “transmission intensity” used herein means the percentage of light travelling through an object. For example, in embodiments of the present disclosure, it refers to the percentage of light travelling through a glass vial containing the diisocyanate composition.

As mentioned above, diisocyanates are important intermediates in many organic synthesis, for example, for preparing polyurethane resins. However, diisocyanates have very reactive groups which make it easily get self-polymerized and thereby become turbid during storage. These problems exist in various diisocyanates including aliphatic, cycloaliphatic and aromatic diisocyanates.

I. Diisocyanate Stabilizer In view of the existing difficulties in stabilizing diisocyanates in prior art, in a first aspect, the present disclosure provides a diisocyanate stabilizer, comprising a sterically hindered phenol other than butylated hydroxytoluene, a thioether and a phosphite other than triphenyl phosphite.

Inventors of the present disclosure surprisingly found that sterically hindered phenol, thioether and phosphite can work synergistically to effectively prevent diisocyanate from self-polymerization. By combining sterically hindered phenol, thioether and phosphite together, the present disclosure provides a stabilizer, which can prevent diisocyanate from turning yellow and/or becoming turbid during long-term storage and heating condition (e.g. melting process having a temperature of around 100° C.). The stabilizer of the present disclosure has effectively solved the problems that the diisocyanates are easy to turn yellow and become turbid during long-term storage and heating condition.

### Sterically Hindered Phenol

The sterically hindered phenol includes phenols that have one or more phenolic hydroxyl groups on the aromatic ring, and preferably those that have a substituent, preferably an alkyl group, in the ortho positions, most preferably in the ortho and para positions, to the phenolic hydroxyl group(s).

It should be understood that above description is not intended to limit the scope of sterically hindered phenols that are suitable for the present disclosure.

Examples of suitable phenols include alkylphenols, for example, o-, m- or p-cresol (methylphenol), 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol, or 2,2′-methylenebis(6-tert-butyl-4-methylphenol), 4,4′-oxydiphenol, 3,4-methylenedioxydiphenol (sesamol), 3,4-dimethylphenol, hydroquinone, tert-butylhydroquinone, 2,5-di-tert-butylhydroquinone, 2-methyl-p-hydroquinone, 2,3-dimethylhydroquinone, trimethylhydroquinone, pyrocatechol (1,2-dihydroxybenzene), 2-(1′-methylcyclohex-1′-yl)-4,6-dimethylphenol, 2- or 4-(1′-phenyleth-1′-yl)phenol, 2-tert-butyl-6-methylphenol, 2,4,6-tris-tert-butylphenol, 2,6-di-tert-butylphenol, nonylphenol [11066-49-2], octylphenol [140-66-9], 2,6-dimethylphenol, bisphenol A, bisphenol F, bisphenol B, bisphenol C, bisphenol S, 3,3′,5,5′-tetrabromobisphenol A, Koresin® from BASF AG, methyl 3,5-di-tert-butyl-4-hydroxybenzoate, 4-tert-butylpyrocatechol, 2-hydroxybenzyl alcohol, 2-methoxy-4-methylphenol, 2,3,6-trimethylphenol, 2,4,5-trimethylphenol, 2,4,6-trimethylphenol, 2-isopropylphenol, 4-isopropylphenol, 6-isopropyl-m-cresol, n-octadecyl β-(3,5-di-tert-butyl-4-hydroxyphenyl)propionate, 1,1,3-tris-(2-methyl-4-hydroxy-5-tert-butylphenyl)butane, 1,3,5-trimethyl-2,4,6-tris(3,5-di-tert-butyl-4-hydroxybenzyl)benzene, 1,3,5-tris-(3,5-di-tert-butyl-4-hydroxybenzyl) isocyanurate, 1,3,5,-tris-(3,5-di-tert-butyl-4-hydroxyphenyl)propionyloxyethyl isocyanurate, 1,3,5-tris-(2,6-dimethyl-3-hydroxy-4-tert-butylbenzyl) isocyanurate or pentaerythritol tetrakis[β-(3,5,-di-tert-butyl-4-hydroxyphenyl)propionate], 2,6-di-tert-butyl-4-dimethylaminomethylphenol, 6-isobutyl-2,4-dinitrophenol, 6-sec-butyl-2,4-dinitrophenol, 2,6-ditert-butyl-4-(4,6-bis(octylthio)-1,3,5-triazin-2-ylamino) phenol, octadecyl 3-(3′,5′-di-tert-butyl-4′-hydroxyphenyl)propionate, hexadecyl 3-(3′,5′-di-tert-butyl-4′-hydroxyphenyl)propionate, octyl 3-(3′,5′-di-tert-butyl-4′-hydroxyphenyl)propionate, 3-thia-1,5-pentanediol bis[(3′,5′-di-tert-butyl-4′-hydroxyphenyl)propionate], 4,8-dioxa-1,11-undecanediol bis[(3′,5′-di-tert-butyl-4′-hydroxyphenyl)propionate], 4,8-dioxa-1,11-undecanediol bis[(3′-tert-butyl-4′-hydroxy-5′-methylphenyl)propionate], 1,9-nonanediol bis[(3′,5′-di-tert-butyl-4′-hydroxyphenyl)propionate], 1,7-heptanediaminebis[3-(3′,5′-di-tert-butyl-4′-hydroxyphenyl)propionamide], 1,1-methanediaminebis[3-(3′,5′-di-tert-butyl-4′-hydroxyphenyl)propionamide], 3-(3′,5′-di-tert-butyl-4′-hydroxyphenyl)propionoic hydrazide, 3-(3′,5′-dimethyl-4′-hydroxyphenyl)propionoic hydrazide, bis(3-tert-butyl-5-ethyl-2-hydroxyphen-1-yl)methane, bis(3,5-di-tert-butyl-4-hydroxyphen-1-yl)methane, bis[3-(1′-methylcyclohex-1′-yl)-5-methyl-2-hydroxyphen-1-yl]methane, bis(3-tertbutyl-2-hydroxy-5-methylphen-1-yl)methane, 1,1-bis(5-tert-butyl-4-hydroxy-2-methylphen-1-yl)ethane, bis(5-tert-butyl-4-hydroxy-2-methylphen-1-yl) sulfide, bis(3-tert-butyl-2-hydroxy-5-methylphen-1-yl) sulfide, 1,1-bis(3,4-dimethyl-2-hydroxyphen-1-yl)-2-methylpropane, 1,1-bis(5-tert-butyl-3-methyl-2-hydroxyphen-1-yl)butane, 1,3,5-tris[1′-(3″,5″-di-tert-butyl-4″-hydroxyphen-1″-yl)-meth-1′-yl]-2,4,6-trimethylbenzene, 1,1,4-tris(5′-tert-butyl-4′-hydroxy-2′-methylphen-1′-yl)butane, aminophenols, such as para-aminophenol, 3-diethylaminophenol, nitrosophenols, such as para-nitrosophenol, p-nitroso-o-cresol, alkoxyphenols, such as 2-methoxyphenol (Guajacol, pyrocatechol monomethyl ether), 4-methoxyphenol (hydroquinone monomethyl ether), 2-ethoxyphenol, 4-ethoxyphenol, 2-isopropoxyphenol, 4-butoxyphenol, mono- or di-tertbutyl-4-methoxyphenol, 3,5-di-tert-butyl-4-hydroxyanisole, 3-hydroxy-4-methoxybenzyl alcohol, 2,5-dimethoxy-4-hydroxybenzyl alcohol (syringa alcohol), 4-hydroxy-3-methoxybenzaldehyde (vanillin), 4-hydroxy-3-ethoxybenzaldehyde (ethyl vanillin), 3-hydroxy-4-methoxybenzaldehyde (isovanillin), 1-(4-hydroxy-3-methoxyphenyl)ethanone (acetovanillon), eugenol, dihydroeugenol, isoeugenol, tocopherols, such as α-, β-, γ-, δ- and ε-tocopherol, tocol, α-tocopherolhydroquinone, 4-methylpyrocatechol, 3-methylpyrocatechol, hydroquinone monobenzyl ether, p-phenoxyphenol, 2,5-di-tert-amylhydroquinone, 2,3-dihydro-2,2-dimethyl-7-hydroxybenzofuran (2,2-dimethyl-7-hydroxycoumaran), 6-hydroxy-2,5,7,8-tetramethylchroman-2-carboxylic acid (Trolox®), and derivatives thereof.

Corresponding sterically hindered phenol products are available, for example, under the trade names Irganox® (BASF), such as pentaerythritol tetrakis[β-(3,5,-di-tert-butyl-4-hydroxyphenyl)propionate] (e.g., Irganox® 1010), 1,3,5-tris(3,5-di-tert-butyl-4-hydroxybenzyl)-1,3,5-triazine-2,4,6(1H,3H,5H)trione (e.g., Irganox® 3114), thiodiethylenebis[3-[3,5-di-tert-butyl-4-hydroxyphenyl]propionate] (e.g., Irganox® 1035), 3,5-bis(1,1-dimethylethyl)-4-benzenepropanoic acid, branched C₇-C₉-alkyl ester (e.g., Irganox® 1135), octadecyl 3-(3,5-ditert-butyl-4-hydroxyphenyl) propionate (e.g., Irganox® 1076), esters of polyglycol ether terminated with phenol derivatives (e.g., Irganox® 2000), 4,6-bis(octylthiomethyl)-o-cresol (e.g., Irganox® 1520); 3,5-bis(1,1-dimethylethyl)-4-hydroxy-benzenepropanoic acid, branched C₁₃-C₁₅-alkyl esters (e.g., Anox® 1315).

It should be understood that above mentioned examples of sterically hindered phenol are merely illustrative and are not intended to limit the protection scope of the present disclosure. Any other suitable sterically hindered phenols are also applicable.

In some preferable embodiments, the sterically hindered phenols include those having a melting point lower than 55° C., for example, sterically hindered phenols having a melting point lower than 20° C., 25° C., 30° C., 40° ° C., 45° C., 50° C. or 55° C. Such sterically hindered phenols enable the stabilizer being liquid at operational temperature (typically at 45° ° C. to 50° C.) and thereby facilitate automatic production.

In preferable embodiments, the sterically hindered phenol is at least one selected from a group consisting of 3,5-bis(1,1-dimethylethyl)-4-benzenepropanoic acid, branched C₇-C₉-alkyl ester (e.g., Irganox® 1135), octadecyl 3-(3,5-di-tert-butyl-4-hydroxyphenyl) propionate (e.g., Irganox® 1076), 3,5-bis(1,1-dimethylethyl)-4-hydroxy-benzenepropanoic acid, branched C₁₃-C₁₅-alkyl esters (e.g., Anox® 1315), esters of polyglycol ether terminated with phenol derivatives (e.g., Irganox® 2000), 4,6-bis(octylthiomethyl)-o-cresol (e.g., Irganox® 1520). These preferable sterically hindered phenols are harmless to human and therefore are safer and environmentally friendly.

In more preferable embodiments, the sterically hindered phenol is at least one selected from a group consisting of 3,5-bis(1,1-dimethylethyl)-4-benzenepropanoic acid, branched C₇-C₉-alkyl ester (e.g., Irganox® 1135) and octadecyl 3-(3,5-di-tert-butyl-4-hydroxyphenyl) propionate (e.g., Irganox® 1076). These more preferable sterically hindered phenols are liquid or solid having low melting point which enables the stabilizer being liquid at room temperature or at typical operational temperature (e.g., 45° C.).

Preferably, the sterically hindered phenol does not include butylated hydroxytoluene (BHT) as BHT is high volatile and is thereby restricted in many applications.

### Thioether

Thioether used according to the present disclosure includes compounds which comprise at least one thioether group, i.e. a sulfur atom which is substituted by two identical or different organic substituents. It should be understood that the above description is not intended to limit the scope of thioethers of the present disclosure. The present disclosure has no limitation on the thioether.

Suitable thioethers have a structure of formula (I)

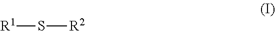

wherein
R¹ and R² can each be, independently of one another, C₁-C₁₈-alkyl, C₂-C₁₈-alkyl which is interrupted by one or more oxygen and/or sulfur atoms and/or one or more substituted or unsubstituted imino groups, C₂-C₁₈-alkenyl, C₆-C₁₂-aryl, C₅-C₁₂-cycloalkyl or a five- or six-membered, oxygen-, nitrogen- and/or sulfur-comprising heterocycle, wherein the radicals mentioned are optionally substituted by hydroxy, amino, aryl, alkyl, aryloxy, alkyloxy, alkylthio, alkoxycarbonyl, nitro, acyl, carbocycles, heteroatoms and/or heterocycles.

Here, R¹ and R² can each be, independently of one another, selected from a group consisting of the following groups:
methyl, ethyl, n-propyl, isopropyl, n-butyl, sec-butyl, tert-butyl, n-pentyl, n-hexyl, n-heptyl, n-octyl, 2-ethylhexyl, 2,4,4-trimethylpentyl, n-decyl, n-dodecyl, n-tetradecyl, n-hetadecyl, n-octadecyl, 1,1-dimethylpropyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonethyl, 2-ethoxycarbonylethyl, 2-butoxycarbonylpropyl, 1,2-di(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, butylthiomethyl, 2-dodecylthioethyl, 2-phenylthioethyl, 2,2,2-trifluoroethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxypropyl, 3-methoxypropyl, 4-methoxybutyl, 6-methoxyhexyl, 2-ethoxypropyl, 3-ethoxypropyl, 4-ethoxybutyl or 6-ethoxyhexyl;
5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-oxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-oxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl;
vinyl, 1-propenyl, allyl, methallyl, 1,1-dimethylallyl, 2-butenyl, 2-hexenyl, octenyl, undecenyl, dodecenyl, octadecenyl, 2-phenylvinyl, 2-methoxyvinyl, 2-ethoxyvinyl, 2-methoxyallyl, 3-methoxyallyl, 2-ethoxyallyl, 3-ethoxyallyl or 1- or 2-chlorovinyl;
phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl;
cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl or a saturated or unsaturated bicyclic system such as norbornyl or norbornenyl; and
furyl, thiophenyl, pyrryl, pyridyl, indolyl, benzoxazolyl, dioxolyl, dioxyl, benzimidazolyl, benzthiazolyl, dimethylpyridyl, methylquinolyl, dimethylpyrryl, methoxyfuryl, dimethoxypyridyl, difluoropyridyl, methylthiophenyl, isopropylthiophenyl or tert-butylthiophenyl.

In some embodiments, the thioether is selected from a group consisting of 2-methyl-1-propenyl tert-dodecyl thioether, cyclohexylidenemethyl n-dodecyl thioether, 3-cyclohexen-(1)ylidenemethyl-n-octadecyl thioether, 3-cyclohexen-(1)-ylidenemethyl-n-dodecyl thioether, 3-cyclohexen-(1)-ylidenemethyl n-octyl thioether, 3-cyclohexen-(1)-ylidenemethyl cyclohexyl thioether, 3-methyl-(3)-cyclohexen-(1)-ylidenemethyl n-dodecyl thioether, 3-cyclohexen-(1)ylidenemethyl p-tolyl thioether, 3-cyclohexen-(1)-ylidenemethyl benzyl thioether and preferably 3-cyclohexen-(1)-ylidenemethyl n-dodecyl thioether and 1-hexenyl-n-dodecyl thioether.

In other embodiments, thioethers are compounds that have a structure of formula (II)

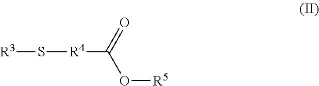

wherein
R³ and R⁵ can each be, independently of one another, C₁-C₁₈-alkyl, C₂-C₁₈-alkyl, which is interrupted by one or more oxygen and/or sulfur atoms and/or one or more substituted or unsubstituted imino groups, C₆-C₁₂-aryl or C₅-C₁₂-cycloalkyl, wherein the radicals mentioned are optionally substituted by hydroxy, amino, aryl, alkyl, aryloxy, alkyloxy, alkylthio, alkoxycarbonyl, nitro, acyl, carbocycles, heteroatoms and/or heterocycles, and
R⁴ can be C₁-C₂₀-alkylene or C₃-C₁₂-cycloalkylene, wherein the radicals mentioned are optionally substituted by hydroxy, amino, aryl, alkyl, aryloxy, alkyloxy, alkylthio, alkoxycarbonyl, nitro, acyl, carbocycles, heteroatoms and/or heterocycles.

Here, R³ and R⁵ can each be, independently of one another, selected from a group consisting of the following groups:
methyl, ethyl, n-propyl, isopropyl, n-butyl, sec-butyl, tert-butyl, n-pentyl, n-hexyl, n-heptyl, n-octyl, 2-ethylhexyl, 2,4,4-trimethylpentyl, n-decyl, n-dodecyl, n-tetradecyl, n-hetadecyl, n-octadecyl, 1,1-dimethylpropyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonethyl, 2-ethoxycarbonylethyl, 2-butoxycarbonylpropyl, 1,2-di(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, butylthiomethyl, 2-dodecylthioethyl, 2-phenylthioethyl, 2,2,2-trifluoroethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxypropyl, 3-methoxypropyl, 4-methoxybutyl, 6-methoxyhexyl, 2-ethoxypropyl, 3-ethoxypropyl, 4-ethoxybutyl or 6-ethoxyhexyl;
5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-oxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-oxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl;
phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl; and cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl or a saturated or unsaturated bicyclic system such as norbornyl or norbornenyl.

Here, R⁴ can be selected from a group consisting of the following groups:
methylene, 1,2-ethylene, 1,2- or 1,3-propylene, 1,2-, 1,3- or 1,4-butylene, 1,1-dimethyl-1,2-ethylene or 1,2-dimethyl-1,2-ethylene, 1,6-hexylene, 1,8-octylene, 1,10-decylene, 1,12-dodecylene and 1,20-eicosylene, and
cyclopropylene, cyclopentylene, cyclohexylene, cyclooctylene and cyclododecylene.

In some preferred embodiments, R³ is optionally substituted C₆-C₁₂-aryl or C₁-C₁₈-alkyl, preferably optionally substituted C₁-C₁₈-alkyl, particularly preferably unsubstituted C₁-C₁₈-alkyl; R⁵ is optionally substituted C₁-C₁₈-alkyl, preferably unsubstituted C₁-C₁₈-alkyl; and/or R⁴ is methylene, 1,2-ethylene or 1,2-propylene, preferably 1,2-ethylene.

In some embodiments, especial preference is given to compounds having a structure of formula (III)

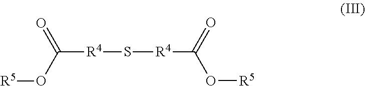

wherein R⁴ and R⁵ are as defined above.

In some preferred embodiments, the thioether includes those having a melting point lower than 50° C., for example, thioethers having a melting point lower than 20° C., 25° C., 30° C., 40° C., 45° C. or 50° C. Such thioethers enable the stabilizer being liquid at operational temperature (typically at 45° ° C. to 50° C.) and thereby facilitate automatic production.

In preferable embodiments, the thioether is at least one selected from a group consisting of thiodipropionic esters, for example dimethyl 3,3′-thiodipropionate, didodecyl 3,3′-thiodipropionate (DLTDP) (e.g., Irganox® PS 800 from BASF), ditridecyl 3,3′-thiodipropionate (DTDTP) (e.g., Songnox® DTDTP from Songwon International AG), dioctadecyl 3,3′-thiodipropionate (DSTDP), dimyristyl thiodipropionate (DMTDP) and 2,2-bis[[3-(dodecylthio)-1-oxopropoxy]methyl]propane-1,3-diyl bis[3-(dodecylthio)propionate] (e.g., Seenox 412S).

In more preferable embodiments, the thioether is at least one selected from a group consisting of didodecyl 3,3′-thiodipropionate (DLTDP) and ditridecyl 3,3′-thiodipropionate (DTDTP). These more preferable thioethers are liquid or solid having a low melting point which enables the stabilizer being liquid at typical operational temperature (e.g., 45° C. to 50° C.).

### Phosphite

Suitable phosphites according to the present disclosure include but not limited to mono phosphites and polymeric phosphites.

Examples of suitable mono phosphites according to the present disclosure include compounds having a structure of formula (IV)

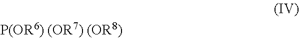

wherein
R⁶, R⁷ and R⁸ can each be, independently of one another, C₁-C₁₈-alkyl, C₆-C₁₂-aryl or C₅-C₁₂-cycloalkyl, wherein the radicals mentioned are optionally substituted by hydroxy, amino, aryl, alkyl, aryloxy, alkyloxy, alkylthio, alkoxycarbonyl, nitro, acyl, carbocycles, heteroatoms and/or heterocycles.

Here, R⁶, R⁷ and R⁸ can each be, independently of one another, selected from a group consisting of the following groups:
methyl, ethyl, n-propyl, isopropyl, n-butyl, sec-butyl, tert-butyl, n-pentyl, n-hexyl, n-heptyl, n-octyl, 2-ethylhexyl, 2,4,4-trimethylpentyl, n-decyl, n-dodecyl, n-tetradecyl, n-hetadecyl, n-octadecyl, 1,1-dimethylpropyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonethyl, 2-ethoxycarbonylethyl, 2-butoxycarbonylpropyl, 1,2-di(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, butylthiomethyl, 2-dodecylthioethyl, 2-phenylthioethyl, 2,2,2-trifluoroethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxypropyl, 3-methoxypropyl, 4-methoxybutyl, 6-methoxyhexyl, 2-ethoxypropyl, 3-ethoxypropyl, 4-ethoxybutyl or 6-ethoxyhexyl;
phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl; and
cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl or a saturated or unsaturated bicyclic system such as norbornyl or norbornenyl.

Examples of suitable polymeric phosphites according to the present disclosure include those having a structure of formula (V)

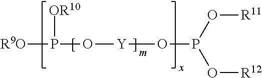

wherein
each R⁹, R¹⁰, R¹¹ and R¹² can be the same or different and independently selected from the group consisting of C1-20 alkyl, C3-22 alkenyl, C6-40 cycloalkyl, C7-40 cycloalkylene, C1-20 methoxy alkyl glycol ethers, C₁-20 alkyl glycol ethers, and/or Y—OH (serving as an end capping moiety);
Y is selected from the group consisting of C₂-40 alkylene, C2-40 alkyl lactone, (e.g., ethylene, propylene, caprylactone), —R¹³—N(R¹⁴)—R¹⁵— (e.g., C2-40 alkyl diamines and C₂-40 alkyl triamines),
wherein R¹³, R¹⁴ and R¹⁵ are independently selected from the group previously defined for R⁹,
R¹⁰, R¹¹ and R¹², now further including H;
m is an integral value ranging from 2 to 100; and
x is an integral value ranging from 1 to 1,000.

In some embodiments, the alkyl, alkenyl, cycloalkyl may be interrupted with oxygen, sulfur or nitrogen.

In some preferable embodiments, the polymeric phosphites are poly(alkylene glycol) phenyl phosphite. More preferably, the poly(alkylene glycol) phenyl phosphite is poly(dipropylene glycol) phenyl phosphite (CAS: 116265-68-0), poly(propylene glycol) phenyl phosphite or poly(ethylene glycol) phenyl phosphite.

In yet other preferable embodiments, the polymeric phosphites are polymeric diphosphites having a structure of formula (VI)

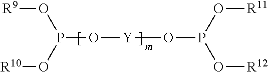

wherein
each R⁹, R¹⁰, R¹¹ and R¹² are as defined previously;
Y is as defined previously; and
m is as defined previously.

Preferably, phosphite according to present disclosure is selected from a group consisting of tridodecyl phosphite (e.g., CCP STAB 3012T), phosphorous acid, mixed 2,4-bis(1,1-dimethylpropyl)phenyl and 4-(1,1-dimethylpropyl)phenyl triesters (CAS: 939402-02-5; e.g. Weston 705), 3,9-bis(isodecyloxy)-2,4,8,10-tetraoxa-3,9-diphosphaspiro[5.5]undecane (e.g., CCP STAB 508T), 4,4′-butylidene-bis(3-methyl6-tert-butylphenyl) alkyl(C₁₃)-phosphite (e.g., CCP STAB AS-4500) and poly(dipropylene glycol) phenyl phosphite (CAS: 116265-68-0). These preferable phosphites are excellent in colour stabilization for diisocyanates during melting process.

More preferably, phosphite is selected from a group consisting of tridodecyl phosphite (e.g., CCP STAB 3012T), phosphorous acid, mixed 2,4-bis(1,1-dimethylpropyl)phenyl and 4-(1,1-dimethylpropyl)phenyl triesters (CAS: 939402-02-5; e.g. Weston 705), 3,9-bis(isodecyloxy)-2,4,8,10-tetraoxa-3,9-diphosphaspiro[5.5]undecane (e.g., CCP STAB 508T) and poly(dipropylene glycol) phenyl phosphite (CAS: 116265-68-0).

Preferably, the phosphite according to present disclosure does not include triphenyl phosphite (TPP) as TPP is restricted by many applications due to its high volatility and toxicity.

In some embodiments, the diisocyanate stabilizer has a melting point of lower than 50° C., preferably lower than 20° C. For example, the diisocyanate stabilizer has a melting point of lower than 50° ° C., 45° C., 40° C., 35° C., 30° C., 25° C., 20° C. or 10° ° C. In some embodiments, the diisocyanate stabilizer is solid at room temperature but is liquid at 45° C.-50° C. When the diisocyanate stabilizer is liquid at operational temperature (e.g., 45° ° C. to 50° C.), it facilitates automatic production, which can effectively improve production efficiency. Further, when the diisocyanate stabilizer has a melting point of lower than 20° C. and is therefore liquid even below 20° C., it additionally facilitates the transportation of the diisocynate stabilizer at low temperature.

Skilled person in the art can obtain a liquid stabilizer having melting point of lower than 50° C. by choosing suitable sterically hindered phenols, thioethers, and/or phosphites according to their melting point and/or by choosing suitable amount of each components. The present disclosure does not make any limitations on those suitable sterically hindered phenols, thioethers, phosphites or the respective amounts in the stabilizer.

For example, in some embodiments, the sterically hindered phenol is at least one selected from a group consisting of 3,5-bis(1,1-dimethylethyl)-4-benzenepropanoic acid, branched C₇-C₉-alkyl ester, octadecyl 3-(3,5-di-tert-butyl-4-hydroxyphenyl) propionate, 3,5-bis(1,1-dimethylethyl)-4-hydroxy-benzenepropanoic acid, branched C₁₃-C₁₅-alkyl esters, esters of polyglycol ether terminated with phenol derivatives, 2,4-dimethyl-6-(1-methylpentadecyl)-phenol and 4,6-bis(octylthiomethyl)-o-cresol; the thioether is at least one selected from a group consisting of didodecyl 3,3′-thiodipropionate, dioctadecyl 3,3′-thiodipropionate, ditridecyl 3,3′-thiodipropionate, dimyristyl thiodipropionate and 2,2-bis[[3-(dodecylthio)-1-oxopropoxy]methyl]propane-1,3-diyl bis[3-(dodecylthio)propionate]; and the phosphite is at least one selected from a group consisting of tridodecyl phosphite, phosphorous acid, mixed 2,4-bis(1,1-dimethylpropyl)phenyl and 4-(1,1-dimethylpropyl)phenyl triesters (CAS: 939402-02-5), 3,9-bis(isodecyloxy)-2,4,8,10-tetraoxa-3,9-diphosphaspiro[5.5]undecane, 4,4′-butylidene-bis(3-methyl6-tert-butylphenyl) alkyl(C₁₃)phosphite and poly(dipropylene glycol) phenyl phosphite (CAS: 116265-68-0). In another embodiments, the sterically hindered phenols include 3,5-bis(1,1-dimethylethyl)-4-benzenepropanoic acid, branched C₇-C₉-alkyl ester and octadecyl 3-(3,5-di-tert-butyl-4-hydroxyphenyl) propionate; the thioethers include didodecyl 3,3′-thiodipropionate and ditridecyl 3,3′-thiodipropionate; and the phosphites include tridodecyl phosphite, phosphorous acid, mixed 2,4-bis(1,1-dimethylpropyl)phenyl and 4-(1,1-dimethylpropyl)phenyl triesters (CAS: 939402-02-5) and 3,9-bis(isodecyloxy)-2,4,8,10-tetraoxa-3,9-diphosphaspiro[5.5]undecane and poly(dipropylene glycol) phenyl phosphite (CAS: 116265-68-0).

In above embodiments, the sterically hindered phenols and thioethers are liquid or solid having a low melting point, which enables the diisocyanate stabilizer being liquid at operational temperature (typically at 45° C. to 50° C.).

As discussed above, the present disclosure has no limitation on the amount of the sterically hindered phenol, thioether, or phosphite. Skilled person in the art may select suitable amounts according to actual needs.

For example, in some embodiments, the sterically hindered phenol is present in an amount of 10 to 90 wt %, the thioether is present in an amount of 5 to 80 wt %, and the phosphite is present in an amount of 5 to 80 wt %, in each case based on the total weight of the stabilizer.

In other embodiments, the sterically hindered phenol is present in an amount of 10 to 90 wt %, e.g., 15 to 85 wt %, 20 to 80 wt %, 25 to 75 wt %, 30 to 70 wt %, 35 to 65 wt %, 40 to 60 wt %, or 45 to 55 wt %. For example, the amount of sterically hindered phenols is 10 wt %, 15 wt %, 20 wt %, 25 wt %, 30 wt %, 35 wt %, 40 wt %, 45 wt %, 55 wt %, 60 wt %, 65 wt %, 70 wt %, 75 wt %, 80 wt %, 85 wt %, 90 wt %, or any value or range of values therebetween.

In yet other embodiments, the thioether is present in an amount of 5 to 80 wt %, e.g., 10 to 75 wt %, or 15 to 70 wt %, or 20 to 65 wt %, or 25 to 60 wt %, or 30 to 55 wt %, or 35 to 50 wt %, or 40 to 45 wt %. For example, the amount of thioethers is 5 wt %, 10 wt %, 15 wt %, 20 wt %, 25 wt %, 30 wt %, 35 wt %, 40 wt %, 45 wt %, 55 wt %, 60 wt %, 65 wt %, 70 wt %, 75 wt %, 80 wt %, or any value or range of values therebetween.

In yet other embodiments, the phosphite is present in an amount of 5 to 80 wt %, e.g., 10 to 75 wt %, or 15 to 70 wt %, or 20 to 65 wt %, or 25 to 60 wt %, or 30 to 55 wt %, or 35 to 50 wt %, or 40 to 45 wt %. For example, the amount of phosphites is 5 wt %, 10 wt %, 15 wt %, 20 wt %, 25 wt %, 30 wt %, 35 wt %, 40 wt %, 45 wt %, 55 wt %, 60 wt %, 65 wt %, 70 wt %, 75 wt %, 80 wt %, or any value or range of values therebetween.

In some preferable embodiments, the diisocyanate stabilizer comprises sterically hindered phenols, thioethers and phosphites, wherein the sterically hindered phenols include 3,5-bis(1,1-dimethylethyl)-4-benzenepropanoic acid, branched C₇-C₉-alkyl ester, octadecyl 3-(3,5-di-tertbutyl-4-hydroxyphenyl) propionate, 3,5-bis(1,1-dimethylethyl)-4-hydroxy-benzenepropanoic acid, branched C₁₃-C₁₅-alkyl esters, esters of polyglycol ether terminated with phenol derivatives, 2,4-dimethyl-6-(1-methylpentadecyl)-phenol and 4,6-bis(octylthiomethyl)-o-cresol; the thioethers include didodecyl 3,3′-thiodipropionate, dioctadecyl 3,3′-thiodipropionate, ditridecyl 3,3′-thiodipropionate, dimyristyl thiodipropionate and 2,2-bis[[3-(dodecylthio)-1-oxopropoxy]methyl]propane-1,3-diyl bis[3-(dodecylthio)propionate]; and the phosphites include tridodecyl phosphite, phosphorous acid, mixed 2,4-bis(1,1-dimethylpropyl)phenyl and 4-(1,1-dimethylpropyl)phenyl triesters (CAS: 939402-02-5), 3,9-bis(isodecyloxy)-2,4,8,10-tetraoxa-3,9-diphosphaspiro[5.5]undecane, 4,4′-butylidene-bis(3-methyl6-tert-butylphenyl) alkyl(C₁₃)phosphite and poly(dipropylene glycol) phenyl phosphite (CAS: 116265-68-0); the sterically hindered phenols are present in an amount of 10 to 90 wt %, the thioethers are present in an amount of 5 to 80 wt %, and the phosphites are present in an amount of 5 to 80 wt %, in each case based on the total weight of the diisocyanate stabilizer.

In yet more preferable embodiments, the diisocyanate stabilizer comprises sterically hindered phenols, thioethers and phosphites, wherein the sterically hindered phenols include 3,5-bis(1,1-dimethylethyl)-4-benzenepropanoic acid, branched C₇-C₉-alkyl ester and octadecyl 3-(3,5-ditert-butyl-4-hydroxyphenyl) propionate; the thioethers include didodecyl 3,3′-thiodipropionate and ditridecyl 3,3′-thiodipropionate; and the phosphites include tridodecyl phosphite, phosphorous acid, mixed 2,4-bis(1,1-dimethylpropyl)phenyl and 4-(1,1-dimethylpropyl)phenyl triesters (CAS: 939402-02-5), poly(dipropylene glycol) phenyl phosphite (CAS: 116265-68-0) and 3,9-bis(isodecyloxy)-2,4,8,10-tetraoxa-3,9-diphosphaspiro[5.5]undecane; the amount of sterically hindered phenols is 10 to 90 wt %, the amount of thioethers is 5 to 80 wt %, and the amount of phosphites is 5 to 80 wt %, in each case based on the total weight of the diisocyanate stabilizer.

Below gives some examples for preparing the diisocyanate stabilizer of the present disclosure. Nevertheless, it should be understood that these examples are only for illustrative purpose. The diisocyanate stabilizer of the present disclosure can be prepared by known processes familiar to skilled person in the art. For example, one can add appropriate sterically hindered phenol, thioether and phosphite of suitable amounts sequentially or simultaneously, then heat and mix the composition for a certain amount of time at a certain temperature. In terms of the mixing process, there are no peculiarities, and conventional mixing techniques and apparatus can be used.

### II. Use of the Diisocyanate Stabilizer

In a second aspect, the present disclosure also provides use of the diisocyanate stabilizer according to the first aspect of the present disclosure for stabilizing diisocyanate.

### III. Diisocyanate Composition

In a third aspect, the present disclosure provides a diisocyanate composition, comprising
(A) a diisocyanate, and
(B) a diisocyanate stabilizer according to the first aspect of the present disclosure.

In some embodiments, the present disclosure provides a diisocyanate composition, comprising
(A) a diisocyanate, and
(B) a diisocyanate stabilizer, comprising a sterically hindered phenol, a thioether, and a phosphite.

### Diisocyanate

Examples of suitable diisocyanate include aliphatic, cycloaliphatic and aromatic diisocyanates.

Examples of suitable aliphatic diisocyanates include tetramethylene diisocyanate, hexamethylene diisocyanate (1,6-diisocyanatohexane), octamethylene diisocyanate, decamethylene diisocyanate, dodecamethylene diisocyanate, tetradecamethylene diisocyanate, derivatives of lysine diisocyanate, trimethylhexane diisocyanate or tetramethylhexane diisocyanate.

Examples of suitable cycloaliphatic diisocyanates include 1,2-, 1,3- or 1,4-diisocyanatocyclohexane, 2,4′- or 4,4′-di(isocyanatocyclohexyl)methane, 1-isocyanato-3,3,5-trimethyl-5-(isocyanatomethylcyclohexane) (isophorone diisocyanate), 1,3- or 1,4-bis(isocyanatomethyl)cyclohexane or 2,4- or 2,6-diisocyanato-1-methyl-cyclohexane.

Examples of suitable aromatic diisocyanates include toluene 2,4- or 2,6-diisocyanate and the isomer mixtures thereof, m- or p-xylylene diisocyanate, 2,4′- or 4,4′-diisocyanatodiphenylmethane (MDI) and the isomer mixtures thereof, phenylene 1,3- or 1,4-diisocyanate, 1-chlorophenylene 2,4-diisocyanate, naphthylene 1,5-diisocyanate, biphenylene 4,4′-diisocyanate, 4,4′-diisocyanato-3,3′-dimethylbiphenyl, 3-methyl-diphenylmethane 4,4′-diisocyanate, tetramethylxylylene diisocyanate, 1,4-diisocyanatobenzene or 4,4′-diisocyanatodiphenyl ether.

The diisocyanate stabilizer of the present disclosure is particularly suitable for stabilizing toluene 2,4- or 2,6-diisocyanate and the isomer mixtures thereof, m- or p-xylylene diisocyanate, 2,4′- or 4,4′-diisocyanatodiphenylmethane and the isomer mixtures thereof, and especial preference to 2,4′- or 4,4′-diisocyanatodiphenylmethane, which is more prone to turn yellow due to its specific structure.

Mixtures of said diisocyanates are also suitable. For example, isophorone diisocyanate is usually in the form of a mixture, specifically a mixture of the cis and trans isomers, generally in a proportion of about 60:40 to 80:20 (w/w), preferably in a proportion of about 70:30 to 75:25, and more preferably in a proportion of approximately 75:25. Dicyclohexylmethane 4,4′-diisocyanate may likewise be in the form of a mixture of the different cis and trans isomers.

For the present disclosure, the diisocyanates can be conventionally obtained by phosgenating the corresponding amines or without the use of phosgene, i.e., by phosgene-free processes.

The diisocyanates which may be used preferably have a content of isocyanate groups (calculated as NCO, molecular weight=42) of from 30 to 50 wt %, based on the diisocyanate.

With regard to sterically hindered phenol, thioether and phosphite in the diisocyanate stabilizer, it should be understood that suitable sterically hindered phenol, thioether and phosphite as well as their amounts in the stabilizer are the same as those described in the first aspect of the present disclosure. Therefore, the suitable sterically hindered phenol, thioether and phosphite as well as their amounts in the stabilizer are not elaborated here again. Below only provides some exemplary sterically hindered phenols, thioethers and phosphites as well as their amounts in the diisocyanate stabilizer that are suitable for the diisocyanate composition of present disclosure.

In some embodiments, the sterically hindered phenol is present in an amount of 10 to 90 wt %, the thioether is present in an amount of 5 to 80 wt %, and the phosphite is present in an amount of 5 to 80 wt %, based on the total weight of the diisocyanate stabilizer.

In some embodiments, the diisocyanate stabilizer consists of a sterically hindered phenol, a thioether and a phosphite.

In some embodiments, the diisocyanate stabilizer has a melting point of lower than 50° C., preferably lower than 20° C.

In some embodiments, the sterically hindered phenol is at least one selected from a group consisting of alkylphenols, bisphenol A, bisphenol F, bisphenol B, bisphenol C, bisphenol S, 3,3′,5,5′-tetrabromobisphenol A, methyl 3,5-di-tert-butyl-4-hydroxybenzoate, 4-tert-butylpyrocatechol, 2-hydroxybenzyl alcohol, 2-methoxy-4-methylphenol, n-octadecyl β-(3,5-ditert-butyl-4-hydroxyphenyl)propionate, 1,1,3-tris-(2-methyl-4-hydroxy-5-tert-butylphenyl)butane, 1,3,5-trimethyl-2,4,6-tris(3,5-di-tert-butyl-4-hydroxybenzyl)benzene, 1,3,5-tris-(3,5-di-tert-butyl-4-hydroxybenzyl) isocyanurate, 1,3,5,-tris-(3,5-di-tert-butyl-4-hydroxyphenyl)propionyloxyethyl isocyanurate, 1,3,5-tris-(2,6-dimethyl-3-hydroxy-4-tert-butylbenzyl) isocyanurate or pentaerythritol tetrakis[β-(3,5,-di-tert-butyl-4-hydroxyphenyl)propionate], 2,6-di-tert-butyl-4-dimethylaminomethylphenol, 6-isobutyl-2,4-dinitrophenol, 6-sec-butyl-2,4-dinitrophenol, 2,6-ditert-butyl-4-(4,6-bis(octylthio)-1,3,5-triazin-2-ylamino) phenol, octadecyl 3-(3′,5′-di-tert-butyl-4′-hydroxyphenyl)propionate, hexadecyl 3-(3′,5′-di-tert-butyl-4′-hydroxyphenyl)propionate, octyl 3-(3′,5′-di-tert-butyl-4′-hydroxyphenyl)propionate, 3-thia-1,5-pentanediol bis[(3′,5′-di-tert-butyl-4′-hydroxyphenyl)propionate], 4,8-dioxa-1,11-undecanediol bis[(3′,5′-di-tert-butyl-4′-hydroxyphenyl)propionate], 4,8-dioxa-1,11-undecanediol bis[(3′-tert-butyl-4′-hydroxy-5′-methylphenyl)propionate], 1,9-nonanediol bis[(3′,5′-di-tert-butyl-4′-hydroxyphenyl)propionate], 1,7-heptanediaminebis[3-(3′,5′-di-tert-butyl-4′-hydroxyphenyl)propionamide], 1,1-methanediaminebis[3-(3′,5′-di-tert-butyl-4′-hydroxyphenyl)propionamide], 3-(3′,5′-di-tert-butyl-4′-hydroxyphenyl)propionoic hydrazide, 3-(3′,5′-dimethyl-4′-hydroxyphenyl)propionoic hydrazide, bis(3-tert-butyl-5-ethyl-2-hydroxyphen-1-yl)methane, bis(3,5-di-tert-butyl-4-hydroxyphen-1-yl) methane, bis[3-(1′-methylcyclohex-1′-yl)-5-methyl-2-hydroxyphen-1-yl]methane, bis(3-tertbutyl-2-hydroxy-5-methylphen-1-yl)methane, 1,1-bis(5-tert-butyl-4-hydroxy-2-methylphen-1-yl)ethane, bis(5-tert-butyl-4-hydroxy-2-methylphen-1-yl) sulfide, bis(3-tert-butyl-2-hydroxy-5-methylphen-1-yl) sulfide, 1,1-bis(3,4-dimethyl-2-hydroxyphen-1-yl)-2-methylpropane, 1,1-bis(5-tert-butyl-3-methyl-2-hydroxyphen-1-yl)butane, 1,3,5-tris[1′-(3″,5″-di-tert-butyl-4″-hydroxyphen-1″-yl)-meth-1′-yl]-2,4,6-trimethylbenzene, 1,1,4-tris(5′-tert-butyl-4′-hydroxy-2′-methylphen-1′-yl)butane, aminophenols, nitrosophenols, alkoxyphenols, eugenol, dihydroeugenol, isoeugenol, tocopherols, tocol, α-tocopherolhydroquinone, 4-methylpyrocatechol, 3-methylpyrocatechol, hydroquinone monobenzyl ether, p-phenoxyphenol, hydroquinone, tert-butylhydroquinone, 2,5-di-tert-butylhydroquinone, 2-methyl-p-hydroquinone, 2,3-dimethylhydroquinone, trimethylhydroquinone, 2,5-di-tert-amylhydroquinone, 2,3-dihydro-2,2-dimethyl-7-hydroxybenzofuran (2,2-dimethyl-7-hydroxycoumaran), 6-hydroxy-2,5,7,8-tetramethylchroman-2-carboxylic acid, and derivatives thereof.

In some embodiments, the sterically hindered phenol is at least one selected from a group consisting of 3,5-bis(1,1-dimethylethyl)-4-benzenepropanoic acid, branched C₇-C₉-alkyl ester, octadecyl 3-(3,5-di-tert-butyl-4-hydroxyphenyl) propionate, 3,5-bis(1,1-dimethylethyl)-4-hydroxy-benzenepropanoic acid, branched C₁₃-C₁₅-alkyl esters, esters of polyglycol ether terminated with phenol derivatives, 2,4-dimethyl-6-(1-methylpentadecyl)-phenol and 4,6-bis(octylthiomethyl)-o-cresol.

In some embodiments, the thioether is at least one selected from formula (III)

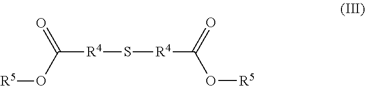

wherein
R⁴ is selected from C₁-C₂₀-alkylene or C₃-C₁₂-cycloalkylene, optionally substituted by hydroxy, amino, aryl, alkyl, aryloxy, alkyloxy, alkylthio, alkoxycarbonyl, nitro, acyl, carbocycles, heteroatoms and/or heterocycles, and
R⁵ is selected from C₁-C₁₈-alkyl, C₂-C₁₈-alkyl, which is interrupted by one or more oxygen and/or sulfur atoms and/or one or more substituted or unsubstituted imino groups, C₆-C₁₂-aryl or C₅-C₁₂-cycloalkyl, which are optionally substituted by hydroxy, amino, aryl, alkyl, aryloxy, alkyloxy, alkylthio, alkoxycarbonyl, nitro, acyl, carbocycles, heteroatoms and/or heterocycles.

In some embodiments, the thioether is at least one selected from a group consisting of didodecyl 3,3′-thiodipropionate, dioctadecyl 3,3′-thiodipropionate, ditridecyl 3,3′-thiodipropionate, dimyristyl thiodipropionate and 2,2-bis[[3-(dodecylthio)-1-oxopropoxy]methyl]propane-1,3-diyl bis[3-(dodecylthio) propionate].

In some embodiments, the phosphite is at least one selected from formula (IV)

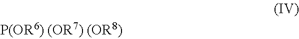

wherein
R⁶, R⁷ and R⁸ are each independently selected from C₁-C₁₈-alkyl, C₆-C₁₂-aryl or C₅-C₁₂-cycloalkyl, which are optionally substituted by hydroxy, amino, aryl, alkyl, aryloxy, alkyloxy, alkylthio, alkoxycarbonyl, nitro, acyl, carbocycles, heteroatoms and/or heterocycles.

In some embodiments, the phosphite is polymeric phosphites having a structure of formula (V)

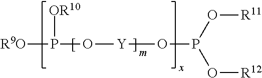

wherein
each R⁹, R¹⁰, R¹¹ and R¹² can be the same or different and independently selected from the group consisting of C1-20 alkyl, C₃-22 alkenyl, C6-40 cycloalkyl, C7-40 cycloalkylene, C1-20 methoxy alkyl glycol ethers, C1-20 alkyl glycol ethers, and/or Y—OH (serving as an end capping moiety);
Y is selected from the group consisting of C2-40 alkylene, C2-40 alkyl lactone, (e.g., ethylene, propylene, caprylactone), —R¹³—N(R¹⁴)—R¹⁵— (e.g., C2-40 alkyl diamines and C2-40 alkyl triamines),
wherein R¹³, R¹⁴ and R¹⁵ are independently selected from the group previously defined for R⁹,
R¹⁰, R¹¹ and R¹², now further including H;
m is an integral value ranging from 2 to 100; and
x is an integral value ranging from 1 to 1,000.

In some embodiments, the polymeric phosphites are polymeric diphosphites having a structure of formula (VI)

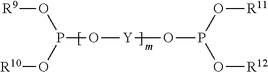

wherein
each R⁹, R¹⁰, R¹¹ and R¹² are as defined previously;
Y is as defined previously; and
m is as defined previously.

In some embodiments, the phosphite is at least one selected from a group consisting of tridodecyl phosphite, phosphorous acid, mixed 2,4-bis(1,1-dimethylpropyl)phenyl and 4-(1,1-dimethylpropyl)phenyl triesters (CAS: 939402-02-5), 3,9-bis(isodecyloxy)-2,4,8,10-tetraoxa-3,9-diphosphaspiro[5.5]undecane, 4,4′-butylidene-bis(3-methyl6-tert-butylphenyl) alkyl(C₁₃)phosphite, and poly(dipropylene glycol) phenyl phosphite (CAS: 116265-68-0).

In some embodiments, the diisocyanate stabilizer comprises sterically hindered phenols, thioethers and phosphites, wherein the sterically hindered phenols include 3,5-bis(1,1-dimethylethyl)-4-benzenepropanoic acid, branched C₇-C₉-alkyl ester, octadecyl 3-(3,5-di-tertbutyl-4-hydroxyphenyl) propionate, 3,5-bis(1,1-dimethylethyl)-4-hydroxy-benzenepropanoic acid, branched C₁₃-C₁₅-alkyl esters, esters of polyglycol ether terminated with phenol derivatives, 2,4-dimethyl-6-(1-methylpentadecyl)-phenol and 4,6-bis(octylthiomethyl)-o-cresol; the thioethers include didodecyl 3,3′-thiodipropionate, dioctadecyl 3,3′-thiodipropionate, ditridecyl 3,3′-thiodipropionate, dimyristyl thiodipropionate and 2,2-bis[[3-(dodecylthio)-1-oxopropoxy]methyl]propane-1,3-diyl bis[3-(dodecylthio)propionate]; and the phosphites include tridodecyl phosphite, phosphorous acid, mixed 2,4-bis(1,1-dimethylpropyl)phenyl and 4-(1,1-dimethylpropyl)phenyl triesters (CAS: 939402-02-5), poly(dipropylene glycol) phenyl phosphite (CAS: 116265-68-0), 3,9-bis(isodecyloxy)-2,4,8,10-tetraoxa-3,9-diphosphaspiro[5.5]undecane, 4,4′-butylidene-bis(3-methyl6-tert-butylphenyl) alkyl(C₁₃)-phosphite; the sterically hindered phenols are present in an amount of 10 to 90 wt %, the thioethers are present in an amount of 5 to 80 wt %, and the phosphites are present in an amount of 5 to 80 wt %, in each case based on the total weight of the diisocyanate stabilizer.

In some embodiments, the diisocyanate stabilizer comprises sterically hindered phenols, thioethers and phosphites, wherein the sterically hindered phenols include 3,5-bis(1,1-dimethylethyl)-4-benzenepropanoic acid, branched C₇-C₉-alkyl ester and octadecyl 3-(3,5-ditert-butyl-4-hydroxyphenyl) propionate; the thioethers include didodecyl 3,3′-thiodipropionate and ditridecyl 3,3′-thiodipropionate; and the phosphites include tridodecyl phosphite, phosphorous acid, mixed 2,4-bis(1,1-dimethylpropyl)phenyl and 4-(1,1-dimethylpropyl)phenyl triesters (CAS: 939402-02-5), poly(dipropylene glycol) phenyl phosphite (CAS: 116265-68-0) and 3,9-bis(isodecyloxy)-2,4,8,10-tetraoxa-3,9-diphosphaspiro[5.5]undecane; the amount of sterically hindered phenols is 10 to 90 wt %, the amount of thioethers is 5 to 80 wt %, and the amount of phosphites is 5 to 80 wt %, in each case based on the total weight of the diisocyanate stabilizer.

In some embodiments, the diisocyanate stabilizer is present in an amount of 0.08 to 0.5 wt %, based on the total weight of the diisocyanate composition. In some preferable embodiments, the diisocyanate stabilizer is present in an amount of 0.1 to 0.4 wt %, and more preferably 0.15 to 0.35 wt %, based on the total weight of the diisocyanate composition.

With the above preferred amount range, the diisocyanate stabilizer can impart enough stability to diisocyanates economically and without a negative impact on the appearance of diisocyanates.

The diisocyanate composition can be prepared by common ways in the art. For example, one can prepare the diisocyanate stabilizer according to the first aspect of the present disclosure in advance, add it into the diisocyanate in an appropriate dosage, then mix them for a certain amount of time. Alternatively, one can add certain amount of sterically hindered phenol, thioether and phosphite into the diisocyanate directly in a sequential way or simultaneous way, then mix them for a certain amount of time. The obtained diisocyanate composition can be stored for a prolonged time and at heating condition without affecting its colour and transparency.

The present disclosure is further illustrated by the following examples.

### EXAMPLES

### Materials

Diisocyanate: Monomeric MDI: (4,4′-diisocyanatodiphenylmethane, Lupranate® ME, BASF)
Stabilizer: Sterically hindered phenols:
   3,5-bis(1,1-dimethylethyl)-4-benzenepropanoic acid, branched C₇-C₉-alkyl ester (Irganox® 1135 from BASF)
   octadecyl 3-(3,5-di-tert-butyl-4-hydroxyphenyl) propionate (Irganox® 1076 from BASF SE)
   2,6-di-tert-butyl-4-methylphenol (BHT, SCRC)
   Thioether:
   didodecyl 3,3′-thiodipropionate (DLTDP, Irganox® PS 800 from BASF)
   ditridecyl 3,3′-thiodipropionate (DTDTP, Songnox® DTDTP from Songwon International AG)
   Phosphite:
   tridodecyl phosphite (CCP STAB 3012T, CCP)
   3,9-bis(isodecyloxy)-2,4,8,10-tetraoxa-3,9-diphosphaspiro[5.5]undecane (CCP STAB 508T, CCP)

### Test Methods

Transmission intensity is measured according to the following standard or procedure:

Preparing samples according to below examples and storing the obtained samples in transparent glass vials;

Taking images of each sample by Charge Coupled Device (CCD) camera; and

Calculating light transmission intensity value of each sample according to electron charges tested by CCD camera.

Charge Coupled Device (CCD) is an integrated circuit containing an array of linked, or coupled, capacitors. CCD camera allows for the conversion of incoming photons into electron charges.

The value of electron charges depends on light intensity incident on the CCD. Accordingly light transmission intensity of each samples can be calculated according to electron charges tested by CCD camera.

### Preparation Examples of Diisocyanate Stabilizer (Examples 1-7)

### Example 1

1) Adding 90 g (90 wt %) Irganox® 1135 and 5 g (5 wt %) CCP STAB 3012T in a beaker, mixing well at room temperature;
2) Adding 5 g (5 wt %) DLTDP into the above liquid mixture; and
3) Heating to 45° ° C., keeping the temperature and stirring for 30 minutes, to obtain a diisocyanate stabilizer, which is liquid at room temperature (e.g. 20° C. to 25° C.).

### Example 2

1) Adding 10 g (10 wt %) Irganox® 1135 and 80 g (80 wt %) CCP STAB 3012T in a beaker, mixing well at room temperature;
2) Adding 10 g (10 wt %) DLTDP into the above liquid mixture; and
3) Heating to 45° C., keeping the temperature and stirring for 30 minutes, to obtain a diisocyanate stabilizer, which is liquid at 45° C.

### Example 3

1) Adding 25 g (25 wt %) Irganox® 1135 and 25 g (25 wt %) CCP STAB 3012T in a beaker, mixing well at room temperature;
2) Adding 50 g (50 wt %) DLTDP into the above liquid mixture; and
3) Heating to 45° C., keeping the temperature and stirring for 30 minutes, to obtain a diisocyanate stabilizer, which is liquid at 45° C.

### Example 4

1) Adding 90 g (90 wt %) Irganox® 1076 and 5 g (5 wt %) CCP STAB 3012T in a beaker, mixing well at room temperature;
2) Adding 5 g (5 wt %) DLTDP into the above liquid mixture; and
3) Heating to 45° C., keeping the temperature and stirring for 30 minutes, to obtain a diisocyanate stabilizer, which is liquid at 50° C.

### Example 5

1) Adding 50 g (50 wt %) Irganox® 1135 and 30 g (30 wt %) CCP STAB 508T in a beaker, mixing well at room temperature;
2) Adding 20 g (20 wt %) DLTDP into the above liquid mixture; and
3) Heating to 45° C., keeping the temperature and stirring for 30 minutes, to obtain a diisocyanate stabilizer, which is liquid at 45° C.

### Example 6

1) Adding 10 g (10 wt %) Irganox® 1135 and 10 g (10 wt %) CCP STAB 3012T in a beaker, mixing well at room temperature; and
2) Adding 80 g (80 wt %) DTDTP into the above liquid mixture, stirring for 30 minutes, to obtain a diisocyanate stabilizer, which is liquid at room temperature.

### Example 7

1) Adding 10 g (10 wt %) Irganox® 1135 and 30 g (30 wt %) CCP STAB 3012T in a beaker, mixing well at room temperature; and
2) Adding 60 g (60 wt %) DTDTP into the above liquid mixture, stirring for 30 minutes, to obtain a diisocyanate stabilizer, which is liquid at room temperature.
   Preparation Examples of Diisocyanate Composition (Ex. a to g and Ex. a′ to g′):

The above diisocyanate stabilizers prepared according to Examples 1 to 7 respectively were added into monomeric MDI at 45° C., and mixed well to obtain diisocyanate compositions a to g (containing 0.3 wt % of diisocyanate stabilizer, based on the total weight of the diisocyanate composition) and a′ to g′ (containing 0.1 wt % of diisocyanate stabilizer, based on the total weight of the diisocyanate composition).

Comparative Diisocyanate Composition (Comp. Ex. h and i):

For the purpose of comparison, BHT and DLTDP are respectively added into monomeric MDI at 45° C., and mixed well to obtain diisocyanate compositions. In specific, the diisocyanate composition of Comparative Example h comprises BHT; and the diisocyanate composition of Comparative Example i comprises DLTDP.

Dosages of the diisocyanate stabilizer in each diisocyanate composition, as well as the transmission intensity and appearance of each diisocyanate composition are shown in Tables 1 to 3.

The higher the transmission intensity, the higher the transparency and the less yellowness.

The appearance of diisocyanate compositions was evaluated with scales 1 to 5, in which 1 represents colourless and transparent, and 5 represents turbid and strong yellow. The lower the number, the higher the transparency and the less the yellowness.

TABLE 1
The transmission intensity of each diisocyanate composition
comprising 0.3 wt % dosage of the diisocyanate stabilizer

| | Dosage of | Transmission | Transmission | Transmission |
|---|---|---|---|---|
| | Diisocyanate | intensity | intensity | intensity |
| Diisocyanate | Stabilizer | (Initial, | (45° C., | (100° C., |
| compositions | (wt %) | 45° C.) | 14 days) | 24 hrs) |
| Ex. a | 0.3 | 0.855 | 0.839 | 0.835 |
| Ex. b | 0.3 | 0.847 | 0.813 | 0.815 |
| Ex. c | 0.3 | 0.825 | 0.823 | 0.798 |
| Ex. d | 0.3 | 0.855 | 0.835 | 0.830 |
| Ex. e | 0.3 | 0.838 | 0.823 | 0.838 |
| Ex. f | 0.3 | 0.839 | 0.823 | 0.798 |
| Ex. g | 0.3 | 0.839 | 0.820 | 0.800 |
| Comp. Ex. h | 0.3 | 0.817 | 0.806 | 0.596 |
| Comp. Ex. i | 0.3 | 0.820 | 0.515 | 0.747 |

TABLE 2
The appearance of each diisocyanate composition comprising
0.3 wt % dosage of the diisocyanate stabilizer

| | Dosage of | | | |
|---|---|---|---|---|
| | Diisocyanate | Appearance | Appearance | Appearance |
| Diisocyanate | Stabilizer | (Initial, | (45° C., | (100° C., |
| compositions | (wt %) | 45° C.) | 14 days) | 24 hrs) |
| Ex. a | 0.3 | 1 | 1 | 1 |
| Ex. b | 0.3 | 1 | 1 | 1 |
| Ex. c | 0.3 | 1 | 1 | 1 |
| Ex. d | 0.3 | 1 | 1 | 1 |
| Ex. e | 0.3 | 1 | 1 | 1 |
| Ex. f | 0.3 | 1 | 1 | 1 |
| Ex. g | 0.3 | 1 | 1 | 1 |
| Comp. Ex. h | 0.3 | 1 | 1 | 4 |
| Comp. Ex. i | 0.3 | 1 | 5 | 2 |

TABLE 3
The appearance of each diisocyanate composition comprising
0.1 wt % dosage of the diisocyanate stabilizer

| | Dosage of | | | |
|---|---|---|---|---|
| | Diisocyanate | Appearance | Appearance | Appearance |
| Diisocyanate | Stabilizer | (Initial, | (45° C., | (100° C., |
| compositions | (wt %) | 45° C.) | 14 days) | 24 hrs) |
| Ex. a′ | 0.1 | 1 | 2 | 2 |
| Ex. b′ | 0.1 | 1 | 2 | 1 |
| Ex. c′ | 0.1 | 1 | 2 | 1 |
| Ex. d′ | 0.1 | 1 | 2 | 2 |
| Ex. e′ | 0.1 | 1 | 2 | 1 |
| Ex. f′ | 0.1 | 1 | 2 | 2 |
| Ex. g′ | 0.1 | 1 | 2 | 1 |

As can be seen from Tables 1 to 3, compared to comparative examples h and i, the diisocyanate compositions (Ex. a-g, and Ex. a′-g′) containing the diisocyanate stabilizer according to the present disclosure have excellent stability in terms of both colour and transparency. In specific, the diisocyanate compositions containing the diisocyanate stabilizer according to the present disclosure can keep colourless (light yellow in some examples), as well as transparent (little sedimentation in some examples) after long-term storage (45° C., 14 days) and heating condition (100° ° C., 24 hours).

Although the embodiments and examples of the present disclosure are described above, skilled person in the art should understand that they are only for illustrative purpose, and are not intended to limit the protection scope of the present disclosure. The protection scope of the present disclosure is defined by the appended claims. Skilled person in the art can make various modifications, equivalent substitutions or improvements to these embodiments without departing from the scope and spirit of the present disclosure, but these modifications, equivalent substitutions or improvements fall within the protection scope of the present disclosure.

1. A diisocyanate stabilizer, comprising a sterically hindered phenol other than butylated hydroxytoluene, a thioether, and a phosphite other than triphenyl phosphite.

2. The diisocyanate stabilizer according to claim 1, wherein the sterically hindered phenol is present in an amount of 10 to 90 wt %, the thioether is present in an amount of 5 to 80 wt %, and the phosphite is present in an amount of 5 to 80 wt %, based on the total weight of the diisocyanate stabilizer.

3. The diisocyanate stabilizer according to claim 1, consisting of a sterically hindered phenol, a thioether and a phosphite.

4. The diisocyanate stabilizer according to claim 1, wherein the diisocyanate stabilizer has a melting point of lower than 50° C., preferably lower than 20° C.

5. The diisocyanate stabilizer according to claim 1, wherein the sterically hindered phenol is at least one selected from a group consisting of alkylphenols, bisphenol A, bisphenol F, bisphenol B, bisphenol C, bisphenol S, 3,3′,5,5′-tetrabromobisphenol A, methyl 3,5-di-tert-butyl-4-hydroxybenzoate, 4-tert-butylpyrocatechol, 2-hydroxybenzyl alcohol, 2-methoxy-4-methylphenol, n-octadecyl β-(3,5-di-tert-butyl-4-hydroxyphenyl)propionate, 1,1,3-tris-(2-methyl-4-hydroxy-5-tert-butylphenyl)butane, 1,3,5-trimethyl-2,4,6-tris(3,5-di-tert-butyl-4-hydroxybenzyl)benzene, 1,3,5-tris-(3,5-di-tert-butyl-4-hydroxybenzyl) isocyanurate, 1,3,5,-tris-(3,5-di-tert-butyl-4-hydroxyphenyl)propionyloxyethyl isocyanurate, 1,3,5-tris-(2,6-dimethyl-3-hydroxy-4-tert-butylbenzyl) isocyanurate or pentaerythritol tetrakis[β-(3,5,-di-tert-butyl-4-hydroxyphenyl)propionate], 2,6-di-tert-butyl-4-dimethylaminomethylphenol, 6-isobutyl-2,4-dinitrophenol, 6-sec-butyl-2,4-dinitrophenol, 2,6-ditert-butyl-4-(4,6-bis(octylthio)-1,3,5-triazin-2-ylamino) phenol, octadecyl 3-(3′,5′-di-tert-butyl-4′-hydroxyphenyl)propionate, hexadecyl 3-(3′,5′-di-tert-butyl-4′-hydroxyphenyl)propionate, octyl 3-(3′,5′-di-tert-butyl-4′-hydroxyphenyl)propionate, thiodiethylenebis[3-[3,5-di-tert-butyl-4-hydroxyphenyl]propionate], 4,8-dioxa-1,11-undecanediol bis[(3′,5′-di-tert-butyl-4′-hydroxyphenyl)propionate], 4,8-dioxa-1,11-undecanediol bis[(3′-tert-butyl-4′-hydroxy-5′-methylphenyl)propionate], 1,9-nonanediol bis[(3′,5′-di-tert-butyl-4′-hydroxyphenyl)propionate], 1,7-heptanediaminebis[3-(3′,5′-di-tert-butyl-4′-hydroxyphenyl)propionamide], 1,1-methanediaminebis[3-(3′,5′-di-tert-butyl-4′-hydroxyphenyl)propionamide], 3-(3′,5′-di-tert-butyl-4′-hydroxyphenyl)propionoic hydrazide, 3-(3′,5′-dimethyl-4′-hydroxyphenyl)propionoic hydrazide, bis(3-tert-butyl-5-ethyl-2-hydroxyphen-1-yl)methane, bis(3,5-di-tert-butyl-4-hydroxyphen-1-yl)methane, bis[3-(l′-methylcyclohex-1′-yl)-5-methyl-2-hydroxyphen-1-yl]methane, bis(3-tert-butyl-2-hydroxy-5-methylphen-1-yl)methane, 1,1-bis(5-tert-butyl-4-hydroxy-2-methylphen-1-yl)ethane, bis(5-tert-butyl-4-hydroxy-2-methylphen-1-yl) sulfide, bis(3-tert-butyl-2-hydroxy-5-methylphen-1-yl) sulfide, 1,1-bis(3,4-dimethyl-2-hydroxyphen-1-yl)-2-methylpropane, 1,1-bis(5-tert-butyl-3-methyl-2-hydroxyphen-1-yl)butane, 1,3,5-tris[1′-(3″,5″-di-tert-butyl-4″-hydroxyphen-1″-yl)-meth-1′-yl]-2,4,6-trimethylbenzene, 1,1,4-tris(5′-tertbutyl-4′-hydroxy-2′-methylphen-1′-yl)butane, aminophenols, nitrosophenols, alkoxyphenols, eugenol, dihydroeugenol, isoeugenol, tocopherols, tocol, α-tocopherolhydroquinone, 4-methylpyrocatechol, 3-methylpyrocatechol, hydroquinone monobenzyl ether, p-phenoxyphenol, hydroquinone, tert-butylhydroquinone, 2,5-di-tert-butylhydroquinone, 2-methyl-p-hydroquinone, 2,3-dimethylhydroquinone, trimethylhydroquinone, 2,5-di-tert-amylhydroquinone, 2,3-dihydro-2,2-dimethyl-7-hydroxybenzofuran (2,2-dimethyl-7-hydroxycoumaran), 6-hydroxy-2,5,7,8-tetramethylchroman-2-carboxylic acid, and derivatives thereof.

6. The diisocyanate stabilizer according to claim 1, wherein the sterically hindered phenol is at least one selected from a group consisting of 3,5-bis(1,1-dimethylethyl)-4-benzenepropanoic acid, branched C₇-C₉-alkyl ester, octadecyl 3-(3,5-di-tertbutyl-4-hydroxyphenyl) propionate, 3,5-bis(1,1-dimethylethyl)-4-hydroxy-benzenepropanoic acid, branched C₁₃-C₁₅-alkyl esters, esters of polyglycol ether terminated with phenol derivatives, 2,4-dimethyl-6-(1-methylpentadecyl)-phenol and 4,6-bis(octylthiomethyl)-o-cresol.

7. The diisocyanate stabilizer according to claim 1, wherein the thioether is at least one selected from formula (III) 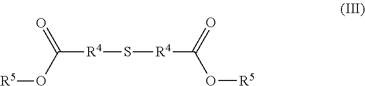
wherein
R⁴ is selected from C₁-C₂₀-alkylene or C₃-C₁₂-cycloalkylene, optionally substituted by hydroxy, amino, aryl, alkyl, aryloxy, alkyloxy, alkylthio, alkoxycarbonyl, nitro, acyl, carbocycles, heteroatoms and/or heterocycles, and
R⁵ is selected from C₁-C₁₈-alkyl, C₂-C₁₈-alkyl, which is interrupted by one or more oxygen and/or sulfur atoms and/or one or more substituted or unsubstituted imino groups, C₆-C₁₂-aryl or C₅-C₁₂-cycloalkyl, which are optionally substituted by hydroxy, amino, aryl, alkyl, aryloxy, alkyloxy, alkylthio, alkoxycarbonyl, nitro, acyl, carbocycles, heteroatoms and/or heterocycles.

8. The diisocyanate stabilizer according to claim 1, wherein the thioether is at least one selected from a group consisting of didodecyl 3,3′-thiodipropionate, dioctadecyl 3,3′-thiodipropionate, ditridecyl 3,3′-thiodipropionate, dimyristyl thiodipropionate and 2,2-bis[[3-(dodecylthio)-1-oxopropoxy]methyl]propane-1,3-diyl bis[3-(dodecylthio)propionate].

9. The diisocyanate stabilizer according to claim 1, wherein the phosphite is at least one selected from formula (IV) 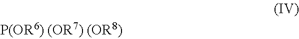
wherein
R⁶, R⁷ and R⁸ are each independently selected from C₁-C₁₈-alkyl, C₆-C₁₂-aryl or C₅-C₁₂-cycloalkyl, which are optionally substituted by hydroxy, amino, aryl, alkyl, aryloxy, alkyloxy, alkylthio, alkoxycarbonyl, nitro, acyl, carbocycles, heteroatoms and/or heterocycles.

10. The diisocyanate stabilizer according to claim 1, wherein the phosphite is polymeric phosphites having a structure of formula (V) 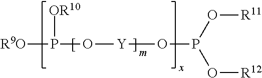
wherein
each R⁹, R¹⁰, R¹¹ and R¹² can be the same or different and independently selected from the group consisting of C1-20 alkyl, C3-22 alkenyl, C6-40 cycloalkyl, C7-40 cycloalkylene, C1-20 methoxy alkyl glycol ethers, C1-20 alkyl glycol ethers, and/or Y—OH (serving as an end capping moiety);
Y is selected from the group consisting of C2-40 alkylene, C2-40 alkyl lactone, (e.g., ethylene, propylene, caprylactone), —R¹³—N(R¹⁴)—R¹⁵— (e.g., C2-40 alkyl diamines and C2-40 alkyl triamines),
wherein R¹³, R¹⁴ and R¹⁵ are independently selected from the group previously defined for R⁹, R¹⁰, R¹¹ and R¹², now further including H;
m is an integral value ranging from 2 to 100; and
x is an integral value ranging from 1 to 1,000.

11. The diisocyanate stabilizer according to claim 10, wherein the polymeric phosphites are polymeric diphosphites having a structure of formula (VI) 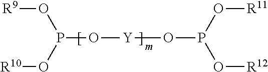
wherein
each R⁹, R¹⁰, R¹¹ and R¹² can be the same or different and independently selected from the group consisting of C1-20 alkyl, C3-22 alkenyl, C6-40 cycloalkyl, C7-40 cycloalkylene, C1-20 methoxy alkyl glycol ethers, C1-20 alkyl glycol ethers, and/or Y—OH (serving as an end capping moiety);
Y is selected from the group consisting of C2-40 alkylene, C2-40 alkyl lactone, (e.g., ethylene, propylene, caprylactone), —R¹³—N(R¹⁴)—R¹⁵— (e.g., C2-40 alkyl diamines and C2-40 alkyl triamines),
wherein R¹³, R¹⁴ and R¹⁵ are independently selected from the group previously defined for R⁹, R¹⁰, R¹¹ and R¹², now further including H; and
m is an integral value ranging from 2 to 100.

12. The diisocyanate stabilizer according to claim 1, wherein the phosphite is at least one selected from a group consisting of tridodecyl phosphite, phosphorous acid, mixed 2,4-bis(1,1-dimethylpropyl)phenyl and 4-(1,1-dimethylpropyl)phenyl triesters (CAS: 939402-02-5), 3,9-bis(isodecyloxy)-2,4,8,10-tetraoxa-3,9-diphosphaspiro[5.5]undecane, 4,4′-butylidene-bis(3-methyl6-tert-butylphenyl) alkyl(C₁₃)-phosphite, and poly(dipropylene glycol) phenyl phosphite (CAS: 116265-68-0).

13. The diisocyanate stabilizer according to claim 1, wherein the sterically hindered phenol is at least one selected from a group consisting of 3,5-bis(1,1-dimethylethyl)-4-benzenepropanoic acid, branched C₇-C₉-alkyl ester, octadecyl 3-(3,5-di-tertbutyl-4-hydroxyphenyl) propionate, 3,5-bis(1,1-dimethylethyl)-4-hydroxy-benzenepropanoic acid, branched C₁₃-C₁₅-alkyl esters, esters of polyglycol ether terminated with phenol derivatives, 2,4-dimethyl-6-(1-methylpentadecyl)-phenol and 4,6-bis(octylthiomethyl)-o-cresol; the thioether is at least one selected from a group consisting of didodecyl 3,3′-thiodipropionate, dioctadecyl 3,3′-thiodipropionate, ditridecyl 3,3′-thiodipropionate, dimyristyl thiodipropionate and 2,2-bis[[3-(dodecylthio)-1-oxopropoxy]methyl]propane-1,3-diyl bis[3-(dodecylthio)propionate]; and the phosphite is at least one selected from a group consisting of tridodecyl phosphite, phosphorous acid, mixed 2,4-bis(1,1-dimethylpropyl)phenyl and 4-(1,1-dimethylpropyl)phenyl triesters (CAS: 939402-02-5), 3,9-bis(isodecyloxy)-2,4,8,10-tetraoxa-3,9-diphosphaspiro[5.5]undecane, 4,4′-butylidene-bis(3-methyl6-tert-butylphenyl) alkyl(C₁₃)phosphite and poly(dipropylene glycol) phenyl phosphite.

14. Use of the diisocyanate stabilizer according to claim 1 Currently Amended for stabilizing diisocyanate.

15. A diisocyanate composition comprising:
(A) a diisocyanate, and
(B) the diisocyanate stabilizer according to claim 1.

16. The diisocyanate composition according to claim 15, wherein the diisocyanate stabilizer is present in an amount of 0.08 to 0.5 wt %, preferably 0.1 to 0.4 wt %, and more preferably 0.15 to 0.35 wt %, based on the total weight of the diisocyanate composition.

17. The diisocyanate composition according to claim 15, wherein the diisocyanate is aromatic diisocyanate.

18. The diisocyanate composition according to claim 17, wherein the aromatic diisocyanate is at least one selected from a group consisting of toluene 2,4- or 2,6-diisocyanate and the isomer mixtures thereof, m- or p xylylene diisocyanate, 2,4′- or 4,4′-diisocyanatodiphenylmethane and the isomer mixtures thereof, phenylene 1,3- or 1,4-diisocyanate, 1-chlorophenylene 2,4 diisocyanate, naphthylene 1,5-diisocyanate, biphenylene 4,4′-diisocyanate, 4,4′-diisocyanato-3,3′-dimethylbiphenyl, 3-methyl-diphenylmethane 4,4′-diisocyanate, tetramethylxylylene diisocyanate, 1,4-diisocyanatobenzene or 4,4′-diisocyanatodiphenyl ether; preferably toluene 2,4- or 2,6-diisocyanate and the isomer mixtures thereof, m- or p-xylylene diisocyanate, 2,4′- or 4,4′-diisocyanatodiphenylmethane and the isomer mixtures thereof.
